(19) United States
(12) Patent Application Publication
(10) Pub. No.: US 2021/0233330 A1
(43) Pub. Date: Jul. 29, 2021

(54) Virtual or Augmented Reality Aided 3D Visualization and Marking System

(71) Applicant: Ottawa Hospital Research Institute, Ottawa (CA)

(72) Inventors: Justin SUTHERLAND, Ottawa (CA); Daniel LA RUSSA, Ottawa (CA)

(73) Assignee: Ottawa Hospital Research Institute, Ottawa, ON (CA)

(21) Appl. No.: 17/250,342

(22) PCT Filed: Jul. 8, 2019
(86) PCT No.: PCT/CA2019/050941

§ 371 Date: Jan. 7, 2021

Related U.S. Application Data
(60) Provisional application No. 62/695,580, filed on Jul. 9, 2018.

(51) Int. Cl. G06T 19/20; G06F 3/0354; G06F 3/041

(52) U.S. Cl. CPC G06T 19/20; G06F 3/03545; G06T 2210/41; G06T 2219/2012; G06F 3/0418

(57) ABSTRACT

There is provided a system and method for applying markings to a three-dimensional virtual image or virtual object, the system comprising a physical stylus, a surface, and a virtual or augmented reality display. A virtual space is displayed by the virtual or augmented reality display. The virtual space includes a three-dimensional target object; at least one plane, including a tracking plane, the tracking plane corresponding to the surface; and a virtual stylus in a virtual reality view, or the physical stylus in an augmented reality view. A position of the virtual stylus or the physical stylus relative to the tracking plane is correlated to an actual position of the physical stylus relative to the surface; and a cross-section of the target object is displayed on the tracking plane where the tracking plane intersects the target object.

### CROSS-REFERENCE TO RELATED APPLICATION(S)

This application claims priority to U.S. Provisional Patent Application No. 62/695,580 filed on Jul. 9, 2018, the contents of which are incorporated herein by reference.

### TECHNICAL FIELD

The following relates to a system and method for visualizing objects and applying markings within a three-dimensional virtual or augmented reality system.

### BACKGROUND

In the medical field, imaging techniques such as computed tomography (CT), magnetic resonance imaging (MRI), positron emission tomography (PET), and other three-dimensional (3D) medical imaging modalities are used to visualize a patient's anatomy. Medical imaging data obtained from these medical imaging procedures can be analyzed to identify organs or other structures of interest, and can be reviewed by a medical professional to determine a diagnosis or appropriate treatment for a patient. For example, radiation oncologists and other radiotherapy clinicians may analyze the medical imaging data to plan a course of radiotherapy treatment and assess the calculated dose to radiotherapy targets.

Traditionally, the medical imaging data of a 3D patient obtained through one or more imaging procedures are presented to medical professionals on screens as digital two-dimensional (2D) slices, or cross-sections. To process the imaging data, the medical professional selects a slice of the scan data along a cardinal plane and draws on the slice using a mouse and cursor or a touch-screen. For example, the slice may show a cross-sectional view of the three-dimensional structure, including a cross-section of any organs or other structures of interest within the three-dimensional structure. The medical professional can mark the image to highlight features of medical importance, draw an outline around (contour) one or more of the organs or other structures, or otherwise annotate the cross-sectional image. This process is often repeated for multiple slices. Outlines of an organ or structure on the multiple slices can be combined to form a 3D contour or model.

Various challenges can arise when using 2D slices to annotate a 3D image volume and/or contour in three dimensions. For example, each 2D image slice is often analyzed and drawn on in isolation, without, or with limited, context and/or knowledge of the orientation and position of the 3D structure. As such, the medical professional may have difficulty identifying organ boundaries on the slice, leading to inaccurate annotations or contours. Additionally, the image slices are often only provided along the three anatomical planes with fixed orientation, namely the sagittal, coronal, and transverse planes. Moreover, certain structures, such as the brachial plexus, are not readily visualized on any of these three conventional planes, thus a medical professional may be unable to accurately identify and/or contour these structures if they are provided with only slices in the three conventional planes.

Three-dimensional imaging data are commonly analyzed on touch-screen computer systems, where the user individually selects 2D slices of the 3D image on which to annotate or contour/draw. The physical dimensions of the touchscreen can create a barrier between the image being drawn upon or annotated and the device used for drawing/annotating, decreasing the precision of annotations or contours. Further, the user's hand and stylus may occlude the line of sight of the image, adding time to the contouring process due to periodic repositioning of the image for visual acuity. Lastly, without knowledge of the 3D shape of a structure, the user may be required to frequently switch between slices, for example to draw on a different slice or provide 3D context, which can be cumbersome and time consuming both for annotating/drawing and when reviewing the contours.

Several systems have been previously proposed to address the above-mentioned shortcomings using virtual reality visualization of the 3D image. These systems allow users to view and interact with 3D models of the imaging data in a virtual space. For example, to create 3D models of organs or structures based on the 3D images represented in a virtual space, a user draws contours by manipulating controllers in the air within the virtual image. These controllers may be uncomfortable to use, since drawing in a virtual space is an unusual and unfamiliar task for most users, and for medical professionals in particular. Additionally, the contours may be imprecise, since there is no physical frame of reference nor direct physical anchor or feedback to indicate to the user where they are drawing in the 3D virtual space. As such, these systems can be cumbersome, difficult to use, and therefore prone to errors and large inter- and intra-user variability in drawing precision.

It is an object of the following to address at least one of the above-noted disadvantages.

### SUMMARY

In one aspect, there is provided a system for applying markings to a three-dimensional virtual image or virtual object, the system comprising: a physical stylus; a surface; and a virtual or augmented reality display; wherein a virtual space is displayed by the virtual or augmented reality display, the virtual space comprising: a three-dimensional target object; at least one plane, including a tracking plane, the tracking plane corresponding to the surface; and a virtual stylus in a virtual reality virtual space, or the physical stylus in an augmented reality virtual space; wherein: a position of the virtual stylus or the physical stylus relative to the tracking plane is correlated to an actual position of the physical stylus relative to the surface; and a cross-section of the target object is displayed on the tracking plane where the tracking plane intersects the target object.

In another aspect, there is provided a method of applying markings to a three-dimensional virtual image or virtual object, the method comprising: displaying a virtual space using a virtual or augmented reality display; providing in the virtual space: a three-dimensional target object, at least one plane including a tracking plane, the tracking plane corresponding to the surface, and a virtual stylus in a virtual reality virtual space, or a physical stylus in an augmented reality virtual space; correlating a position of the virtual stylus or the physical stylus relative to the tracking plane to an actual position of the physical stylus relative to a surface; and displaying a cross-section of the target object on the tracking plane where the tracking plane intersects the target object.

In yet another aspect, there is provided a computer readable medium comprising computer executable instructions for performing the method.

### BRIEF DESCRIPTION OF THE DRAWINGS

Embodiments will now be described with reference to the appended drawings wherein:

FIG. 1 is a pictorial schematic diagram of a virtual reality or augmented reality-aided drawing system;

FIG. 2 is a perspective view of a virtual space displayed on a virtual reality or augmented reality display;

FIG. 3 is a partial perspective view of a 3D object;

FIGS. 4(a) through 4(d) are perspective views of a tracking plane intersecting the 3D object in FIG. 3;

FIGS. 5(a) through 5(c) are perspective views of a tracking plane and a virtual plane intersecting the 3D object;

FIG. 6 is a perspective view of a menu in the virtual space;

FIGS. 7(a) through 7(d) are schematic diagrams illustrating a virtual stylus annotating the 3D object on the tracking plane;

FIG. 8 is a perspective view of 3D contours drawn by the user in relation to a repositioned tracking plane;

FIGS. 9(a) through 9(c) are partial perspective views of the virtual stylus applying one or more markings on a 2D cross-section of the 3D object displayed on the tracking plane;

FIGS. 10(a) and 10(b) are perspective views of the 3D contours partially faded to aid with visualization of the tracking plane;

FIGS. 11(a) and 11(b) are illustrative views of a signed distance field (SDF);

FIG. 12 illustrates projection of a 3D voxel onto 2D SDFs; and

FIG. 13 illustrates example operations performed in contouring using SDFs.

### DETAILED DESCRIPTION

To visualize and apply one or more markings to an object in a virtual 3D space, a virtual or augmented reality system can be utilized, in which the object can be manipulated in a virtual space and such markings (e.g., lines, traces, outlines, annotations, contours, or other drawn or applied marks) can be made on a selected cross-section of the object by moving a physical stylus along a physical surface, which are both also represented in the virtual space.

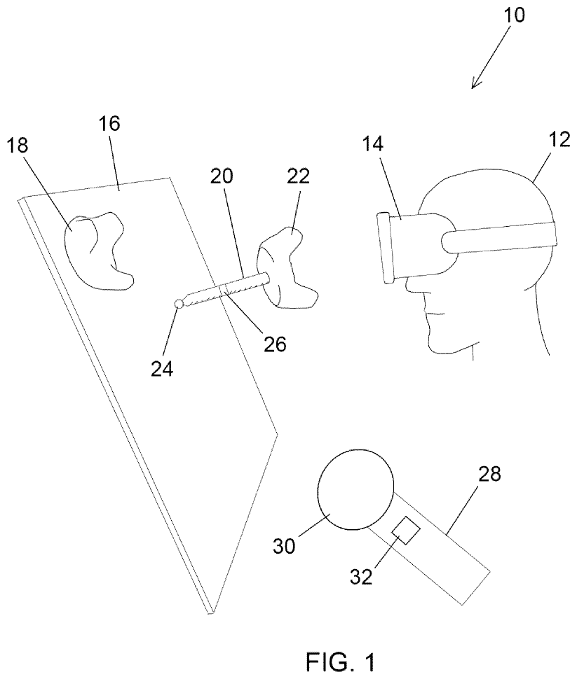

Turning now to the figures, FIG. 1 illustrates a virtual reality- or augmented reality-aided marking system 10 used by a user 12, comprising a surface 16, a stylus 20, and a virtual reality or augmented reality display 14. In this example, the system 10 also includes a controller 28, however in other examples the controller 28 may not be required. The stylus 20 is provided with a first tracker 22. The position of the surface 16 is tracked by the system 10 using, in this example, a second tracker 18 or, in another example, by relating its position to a first tracker 22 via a positional calibration procedure. In one example, the controller 28 is provided with a third tracker 30. The system 10 is configured to determine the position of the display 14 and trackers 18, 22, 30. In this example, the trackers 18, 22, 30 are photodiode circuits such as Vive™ trackers in communication with a laser-sweeping base station (not shown) such that the system 10 can determine the relative location of the display 14, which may include an integrated tracker (not shown), and trackers 18, 22, 30 with respect to the base station. However, it should be appreciated that any other tracking method and/or tracking device can be used, for example optical tracking systems.

The surface 16 can be a rigid or substantially rigid surface of known shape and dimension. In this example, the surface 16 is a rectangle, however the surface 16 may have any other shape such as a circle, triangle, or trapezoid, etc. The surface 16 is illustrated as appreciably planar, however it should be appreciated that the surface 16 may be nonplanar, such as a curved surface or a surface with raised features. In this example, the surface 16 has fixed dimensions, however in other examples, the surface 16 may have adjustable dimensions, such as a frame with telescopic edges placed on a table. In one example, the surface 16 may be a conventional 2D monitor corresponding to the position of the tracking plane which displays a 2D cross-section of the target object, with the system 10 configured to show a perspective view of the target object on the display 14 above the surface 16. The second tracker 18 may be located on a known position on the surface 16, such as a corner of the surface 16. The system 10 is configured to determine the position and orientation of the surface 16 from the position and orientation of the second tracker 18, the known shape and dimension of the surface 16, and the location of the second tracker 18 relative to the surface 16. It may be noted that more than one surface 16 can be provided and interactions with any such additional surface(s) may be tracked using an additional corresponding tracker (not shown). In one embodiment, the system 10 is configured to determine the position and orientation of the surface 16 by way of virtual tracking coordinates that are provided by the user 12 using the stylus 20 to indicate the boundaries of the virtual plane on an existing physical surface 16 such as a tabletop or electronic screen. As described in greater detail below, any one or more surfaces 16 in the physical environment can be represented in the virtual space (which may also be referred to as a virtual “environment”) and used to provide physical interfaces felt by the user 12 while operating the stylus 20 in the physical environment and likewise represented in the virtual space.

The stylus 20 can be a rigid instrument of known dimension held by the user 12 in the manner of a pen or other marking, drawing, writing, etching or interactive instrument, and is used to apply markings such as by drawing contours or annotating the target object and interacting with menus in the virtual space, as described in greater detail below. The stylus 20 is provided with one or more input sensors. In this example, a tip sensor 24 is located on the tip of the stylus 20 and a stylus button 26 is located on the side of the stylus 20. The tip sensor 24 is configured to detect when the tip of the stylus 20 is in contact with the surface 16. The tip sensor 24 may be a capacitive sensor, or a button that is depressed when the stylus 20 is pressed against the surface 16. The tip sensor 24 may also be a photoelectric sensor reflector if the surface 16 is semi-reflective. In some examples, the tip sensor 24 is capable of detecting the force applied by the stylus 20. The stylus button 26 is configured to receive input from the user 12. The stylus button 26 may be a physical button or capacitive sensor to determine a binary state, or the stylus button 26 may be a multi-directional input sensor such as a trackpad or pointing stick. The stylus 20 may also include a haptic motor (not shown) to provide the user 12 with haptic feedback.

The first tracker 22 is located on a known position on the stylus 20, such as on the end opposite of the tip sensor 24. The system 10 is configured to determine the position and orientation of the stylus 20 from the position and orientation of the first tracker 22 using the known dimension of the stylus 20, and the location of the first tracker 22 relative to the stylus 20. It can be appreciated that the stylus tracker 22 depicted in FIG. 1 is illustrative of one particular form-factor, and other form-factors and tracker-types are possible.

The controller 28 is configured to receive input from the user 12, in this example with one or more controller buttons 32. The third tracker 30 is located on a known position on the controller 28. The system 10 is configured to determine the position and orientation of the controller 28 from the position and orientation of the third tracker 30, and the location of the third tracker 30 relative to the controller 28. The controller 28 depicted in FIG. 1 is illustrative of only one particular handheld device and various other form factors and controller-types can be utilized.

In an exemplary use of the system 10, the surface 16 is placed such that it is supported by a table, mount or other supporting structure (not shown for ease of illustration). In another example of the system 10, the surface 16 comprises a table or other rigid surface. The user 12 may manipulate the stylus 20 with a dominant hand, and optionally the controller 28 with the other hand to manipulate images, data, tools, and menus in the virtual space.

The virtual reality or augmented reality display 14 is configured to display a virtual image or virtual object to the user 12. In this example, the display 14 is a virtual reality headset such as the HTC Vive™, and the displayed image is perceived by the user 12 three-dimensionally. While some of the examples described herein are provided in the context of virtual reality, it can be appreciated that the system 10 and principles discussed herein can also be adapted to augmented reality displays, such as see-through glasses that project 3D virtual elements on the view of the real-world. Similarly, the system 10 can use a 3D monitor to provide the surface 16 with shutter glasses to produce a 3D visualization. In general, the display 14 refers to any virtual or augmented reality headset or headgear capable of interacting with 3D virtual elements.

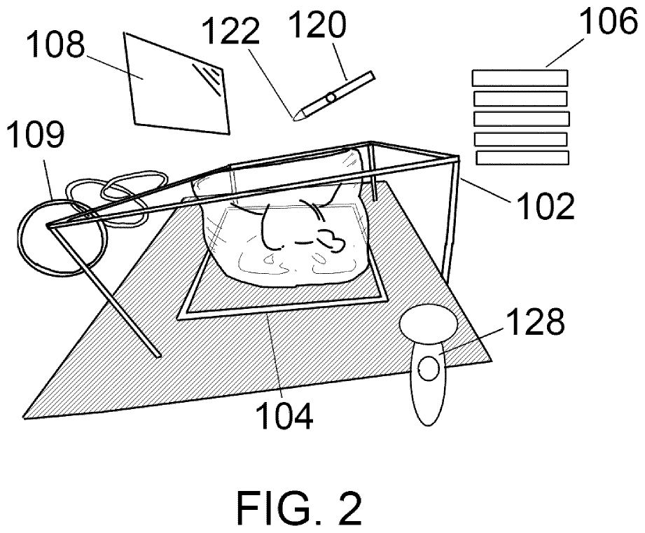

FIG. 2 illustrates an example of a virtual space 100 displayed on the virtual reality or augmented reality display 14. The virtual space 100 comprises a target object 102, a first, primary, or “tracking” plane 104, a menu 106, a virtual stylus 120, and a virtual controller 128. The tracking plane 104 refers to a plane represented in the virtual space that is coupled to or otherwise associated with a physical surface 16 in the physical space. As indicated above, the system 10 can include multiple physical surfaces 16 and, in such cases, would likewise include multiple tracking planes 104 in the virtual space.

The virtual stylus 120 and the virtual controller 128 are virtual, visual representations of the stylus 20 and controller 28, respectively in virtual-reality. It can be appreciated that in augmented-reality examples, the stylus 20 does not have a corresponding virtual stylus 120 in the augmented reality space, which comprises a view of the virtual space 100 and the physical environment. The position of the virtual stylus 120 and the virtual controller 128 in the virtual space 100 are correlated to the physical position of the stylus 20 and the controller 28 determined by the system 10, as described above. In particular, the virtual stylus 120 relative to the tracking plane 104 correlates to the stylus 20 relative to the surface 16 in the physical environment. As such, the user 12 moves or manipulates the virtual stylus 120 and the virtual controller 128 by physically moving or manipulating the stylus 20 and the controller 28, respectively. Similarly, the tracking plane 104 is a virtual representation of the surface 16. The tracking plane 104 has a shape and dimension corresponding to the real dimensions of the surface 16. In this example, the tracking plane 104 is a rectangle with an identical aspect ratio as the physical surface 16. In another example, a portion of the surface 16 may be associated with the tracking plane 104, e.g. the surface 16 boundary is surrounded by a frame or has a handle that is not represented in the virtual space 100. It should be appreciated that if the surface 16 is curved or has raised features, the tracking plane 104 will also have curvature(s) and/or raised feature(s) with the same dimensions as the surface 16. The virtual stylus 120 can be moved towards the tracking plane 104 by moving the stylus 20 towards the surface 16 in the physical environment, and when the tip of the stylus 20 contacts the surface 16, the tip of the virtual stylus 120 contacts the tracking plane 104 in the virtual space 100.

Other objects in the virtual space 100, such as the target object 102 and the menu 106, may be manipulated using the virtual stylus 120 or the virtual controller 128. For example, the user 12 can move the controller 28 such that the virtual controller 128 is over the target object 102, and press one or more of the controller buttons 32 to select the target object 102. In one example, the menu 106 may be manipulated by bringing the virtual stylus 120 within a defined proximity of the menu 106 without the user 12 pressing one or more of the controller buttons 32. In another example (not shown), the menu 106 or a portion thereof can be aligned with the tracking plane 104 to provide tactile feedback when selecting a menu option using the virtual stylus 120. In another example, the menu 106 can be displayed or hidden from view in the virtual space when the user 12 presses one or more of the controller buttons 32. The system 10 may provide the user 12 with feedback to indicate that the target object 102 is available for selection, for example by changing the color of the target object 102 or providing haptic feedback to the controller 28 when the virtual controller 128 is in proximity to the target object 102. With the controller button 32 pressed, the movement of the target object 102 can be associated with the movement of the virtual controller 128, such that movement or rotation of the controller 28 results in corresponding movement or rotation of both the virtual controller 128 and the target object 102. It should be appreciated that the virtual stylus 120 can be similarly used to manipulate the objects in the virtual space.

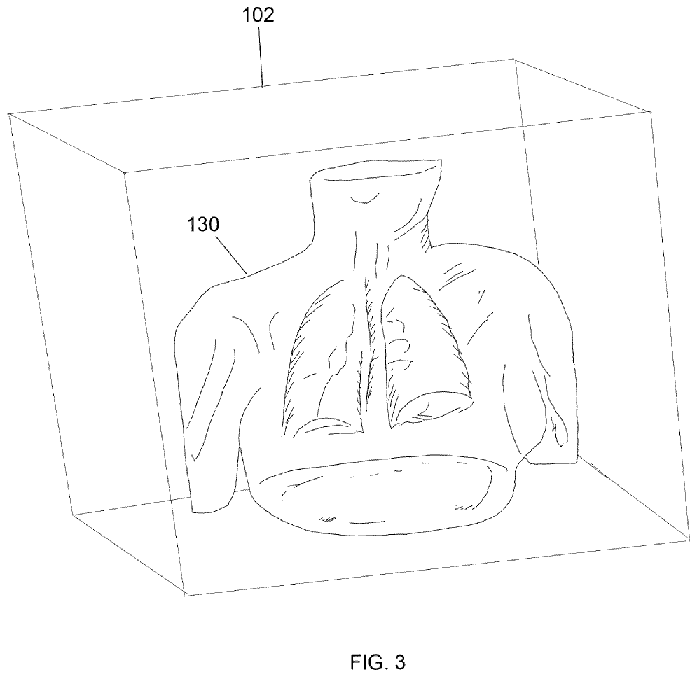

The target object 102 comprises information on a 3D object, for example a medical image, that the user 12 wishes to apply markings to, e.g., to draw on, contour, or annotate, using the system 10. One example of the target object 102 is illustrated in FIG. 3. In this example, the system 10 is used to analyze a patient's medical imaging data, however it should be understood that the system 10 may be used to analyze and/or apply markings to any other 3D object, such as anatomical models or atlases, models of treatment devices or plans, 3D volumetric data, 3D printing designs (e.g., for 3D printing of medical-related models), 3D physiological data, animal medical imaging data or associated models, educational models or video game characters. Further details of various non-medical applications are provided below.

The target object 102 shown in FIG. 3 comprises the 3D object 130 having, in this example, models or medical imaging data of a patient, such as those obtained from CT scans, MRI scans, PET scans, and/or other 3D medical imaging modalities. In this example, the 3D object 130 being analyzed by the system 10 is medical imaging data acquired from CT scan of a human patient, however as noted above, it should be appreciated that the 3D object may consist of medical imaging data or models acquired from any other living being, including animals such as dogs, cats, or birds to name a few. The 3D object 130 can be visualized by performing volume rendering to generate a virtual 3D perspective of the 3D object 130 in the virtual space 100. For example, MRI scan data from a human patient can be volume rendered to create a 3D view of the patient's internal organs. The user 12 can view the 3D object 130 from different angles by rotating or manipulating the target object 102, as described above.

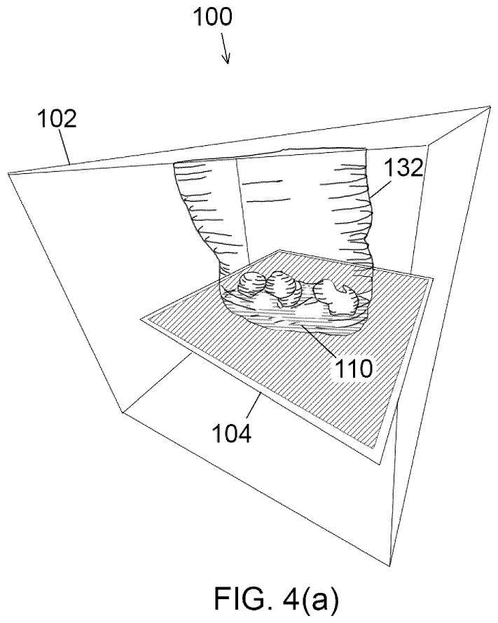

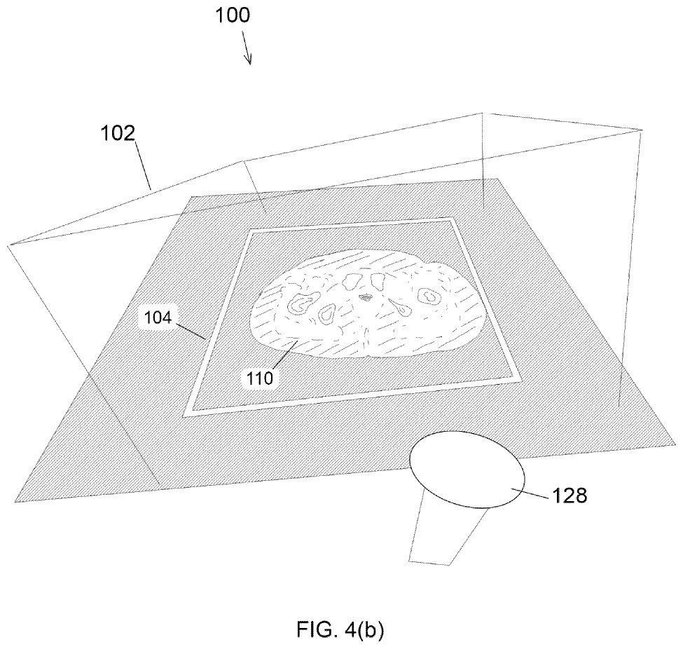

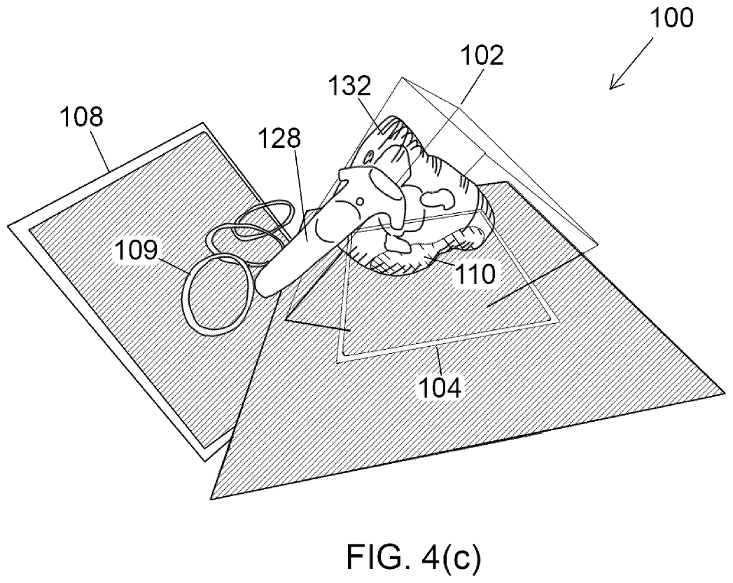

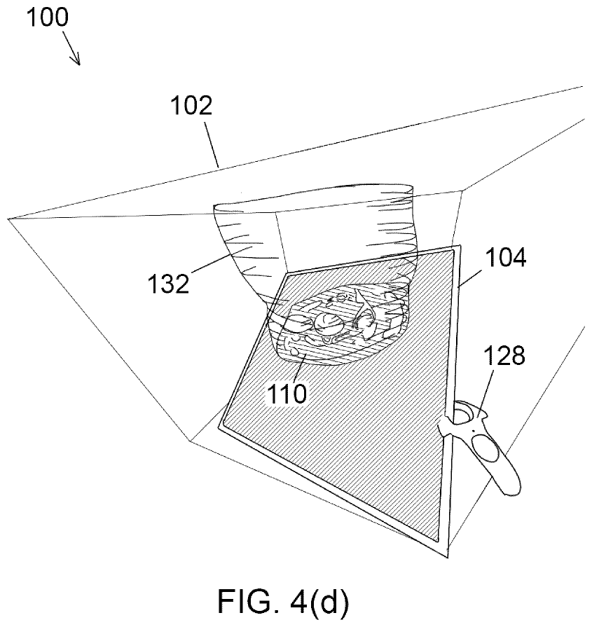

The target object 102 may store other data, including radiotherapy contours 132 (i.e. specific types of markings) around organs or other structures of interest as shown in FIG. 4(a), for example wire-frame contours, 2D or 3D models, 3D color-fill volumes, 3D meshes, or other markings such as annotations, line measurements, points of interest, or text. The radiotherapy contours 132 may be pre-delineated, i.e., generated automatically or by a different user, or drawn by the user 12. The target object 102 is configured to store any information added by the user 12, such as contours drawn or modified around organs or other structures, as described in greater detail below, radiotherapy doses for the contoured organs or structures, or other information, 3D or otherwise, about the target object 102. The user 12 can modify the appearance of the target object 102, for example changing the brightness or hiding the contours 132, by using options in the menu 106, as described in greater detail below.

FIGS. 4(a) through 4(d) show the target object 102 intersected by the tracking plane 104. In FIG. 4(a), the volume-rendered model of the 3D object 130 is hidden from view. The contours 132 drawn by the user 12 are shown in the target object 102, such that the outlines of certain bodily structures are still visible. The contours 132 or volume-rendered 3D object 130 may provide spatial context so the user 12 is aware of the position and orientation of the 3D object 130 as the target object 102 is moved or rotated.

The system 10 is configured to display a cross-section 110 of the target object 102 on the tracking plane 104 derived from the 3D object 130. The underlying 3D object 130 may be stored in a 3D texture (i.e., a collection of voxels in the target object 102). In volume rendering, the system 10 can use a graphics card to trace rays through the 3D texture In order to assign a color and brightness to each pixel on the physical display 14. Along each ray, the associated 3D texture is sampled at regular intervals and color and opacity are accumulated resulting in a final color to display on each individual pixel. The manner by which the accumulation of color and opacity is performed results in the 3D volume being displayed in different ways, the x-ray-like view of FIG. 3 being one example. For the plane 104, the same 3D texture can be used with the system 10 operating the graphics card to display a particular pixel on the cross-section 110. This pixel is shown in the corresponding position to a locale in the 3D texture. As such, the color that is stored in that position in the 3D texture is rendered. In this example, the cross-section 110 shows a 2D cross-section of the 3D object 130 on the plane where the target object 102 is intersected by the tracking plane 104. The cross-section 110 may also display other information such as contours 132, either pre-delineated or drawn by the user 12, or other 3D image objects such as fused imaging data from a separate image acquisitions, 3D physiological data, or radiation doses.

As illustrated in FIG. 4(b), contours 132 (not shown) can be hidden by selecting an option in the menu 106 (not shown), as discussed in greater detail below. The contours 132 may be hidden to provide the user 12 with a clearer view of the cross section 110. The user may also zoom in or increase the size of the target object 102 containing 3D object 130 and subsequently the associated cross section 110, for example by concurrently pressing the stylus button 26 and the controller button 32 while increasing the relative distance between the stylus 20 and the controller 28, or by, for example, selecting zoom options in the menu 106.

FIG. 4(c) shows the user 12 (not physically shown) virtually manipulating the target object 102 using the virtual controller 128 as described above. The user 12 can manipulate the target object 102 to change the plane of intersection with the tracking plane 104, and display a different cross section 110 of the target object 102. As such, the system 10 can generate the cross section 110 along any plane through the 3D object 130, and the user 12 is not limited to cross sections on the three conventional anatomical planes. In medical imaging, this functionality allows the user 12 to view a given anatomical structure on the most anatomically descriptive cross-section. For example, using only the three conventional anatomical planes in medical imaging of human subjects (axial, sagittal, and coronal orientations), the user 12 would have difficultly identifying and visualize the brachial plexis (a bundle of nerves emanating from the spine) but would have no difficultly doing so using the system 10.

The angle and positioning of the tracking plane 104 can be anchored such that the user 12 may move the target object 102 through the tracking plane 104 to display a cross section 110 with a fixed orientation. In another example, the user 12 may anchor an angle of intersection of the target object 102 with the tracking plane 104 by registering two virtual coordinates in space using the virtual stylus 120 or menu 106, around which the 3D object 130, or tracking plane 104, as chosen, can be rotated. Similarly, in another example, the user 12 may choose a single point to anchor rotation of the target object 102 by registering a single virtual coordinate in the virtual space 100 using the virtual stylus 120 or menu 106, such that either the tracking plane 104 or 3D object 130 can rotate around this pivot point.

In another implementation, the target object 102 can be manipulated using only the virtual controller 128 and virtual stylus 120. For example, the user 12 can use the virtual stylus 120 to draw or annotate an axis on cross-section 110 and rotate the target object 102 about the axis defined by this line, by moving the virtual controller 128 into the target object 102, pressing the controller button 32, and moving the virtual controller in a circular motion around the line. In another example, the user 12 can anchor the target object 102 on a point-of-interest to the user 12 on cross-section 110 by placing the virtual stylus tip 122 on the point-of-interest, moving the virtual controller 128 into the target object 102, and moving the virtual controller 128 around the point-of-interest while pressing and holding the controller button 32. Changes to position an (movements left, right, up, or down) or only rotations around pre-defined axes of rotation by, for example, selecting the desired option to rotate or translate in the menu 106 using the virtual stylus 120 or virtual controller 128 in a manner as described above.

In another implementation, the user 12 may store one or more orientations of the target object 102 and tracking plane 104 in the menu 106 such that the user 12 may readily switch between these orientations using menu 106. Similarly, the user 12 may store one or more rotation point-of-interests or axes within the target object 102 for selection in menu 106.

The virtual space 100 may also include one or more object manipulation tools, in this example one or more rotation rings 109, as shown in FIG. 4(c). The user 12 may rotate the target object 102 around an axis of rotation using the one or more rotation rings 109, for example by moving the virtual controller 128 onto the rotation rings 109, pressing the controller button 32, and moving the virtual controller 128 in a circular motion around the periphery of the rotation ring 109. In this example, there are three (3) rotation rings 109, each one corresponding to one of the sagittal, axial, and coronal axes. This functionality may also be accomplished by sliders on a menu or other virtual controller 128 or virtual stylus 120 motions (not shown). The system 10 may also be configured to manipulate the target object 102 when the user 12 presses one or more of the controller buttons 32. For example, one or more of the controller buttons 32 can be configured to cause an incremental movement of the target object 102 in the plane of, or perpendicular to, the tracking plane 104.

In one example, the user 12 can scale (increase or decrease size) the target object 102 and 3D object 130. In one implementation, the user may select an option to scale in the menu 106, and place the virtual controller 128 in the target object, and, while pressing controller button 32, move the virtual controller to scale up or down accordingly. In another implementation, scaling can be with the target object anchored at a point on the cross-section 110 with the virtual stylus tip 122 as described above.

As illustrated in FIG. 4(d), the user 12 can also manipulate the tracking plane 104 to change the plane of intersection between the target object 102 and the tracking plane 104, and thus display a different cross section 110. In this example, the user 12 manipulates the tracking plane 104 in a similar fashion as the target object 102. The user can move the virtual controller 128 over the tracking plane 104, press one or more of the controller buttons 32 to select the tracking plane 104, and move the virtual controller 128 while holding the one or more controller buttons 32. In other examples, the position of the tracking plane 104 is correlated to the physical position of the surface 16 determined by the system 10 using the second tracker 18, as described above. The user 12 can manipulate the surface 16 to move the tracking plane 104, in the same way that the user 12 can manipulate the controller 28 to move the virtual controller 128. If the position of the tracking plane 104 and the surface 16 is decoupled by the user, the user can choose to reorient the tracking plane 104 and target object 102 to realign the tracking plane 104 with the surface 16 by pushing a controller button 32 or menu button 106.

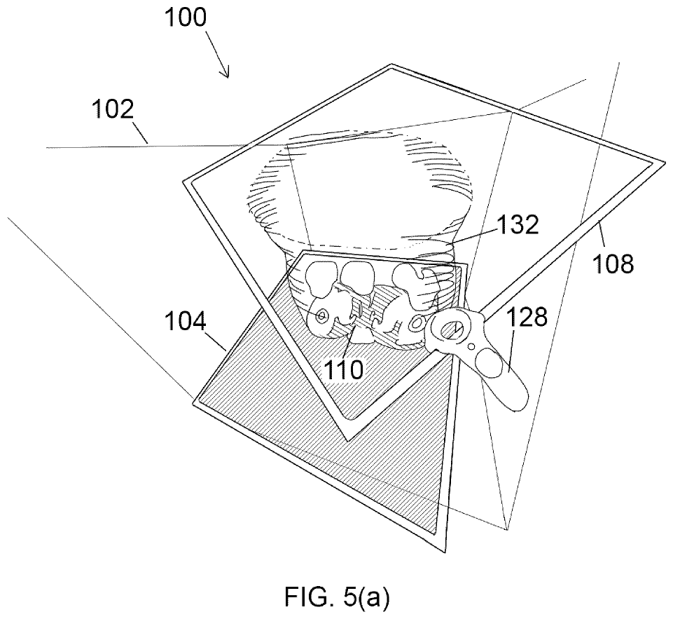

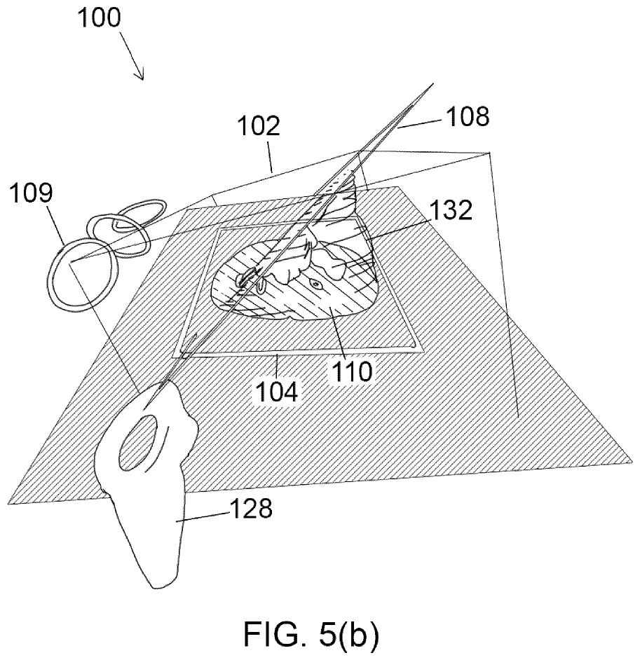

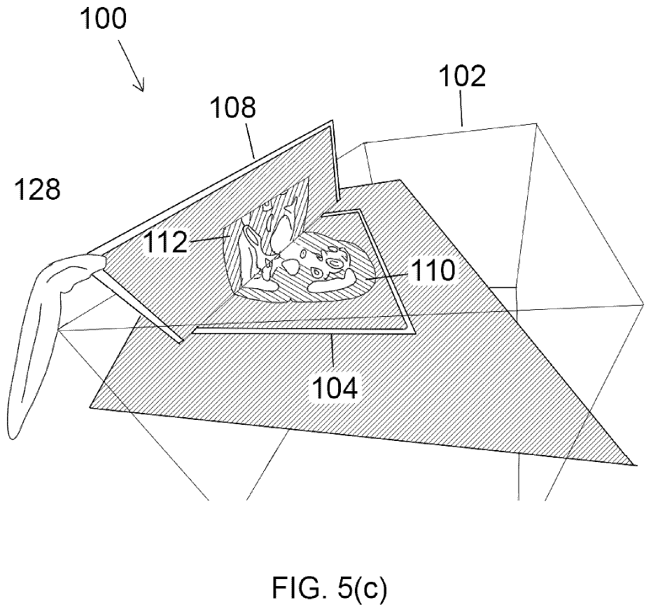

The system 10 may also include within the virtual space 100 one or more virtual planes 108 to change the display of the target object 102. In the example illustrated in FIGS. 5(a) and 5(b), the virtual plane 108 is used to hide the contours 132. As shown in FIG. 5(a), the user 12 can move the virtual plane 108 in a similar fashion as the target object 102. The user can move the virtual controller 128 over the virtual plane 108, press one or more of the controller buttons 32 to select the virtual plane 108, and move the virtual controller 128 while holding the one or more controller buttons 32.

FIG. 5(b) illustrates the virtual plane 108 intersecting the target object 102 at an angle with respect to the tracking plane 104. In this example, the contours 132 are only shown in the volume between the tracking plane 104 and the virtual plane 108. The contours 132 outside of the volume between the tracking plane 104 and the virtual plane 108 are hidden from view. In general, the tracking and virtual planes 104 and 108 may also be considered as, and referred to as, first and second planes, primary and secondary planes, or coupled and uncoupled planes, with the first, primary and coupled nomenclature referring to the plane(s) that is/are associated with the surface 16 in the physical environment, and the second, secondary and uncoupled nomenclature referring to the plane(s) that is/are provided and utilized within the virtual space 100.

FIG. 5(c) illustrates the virtual plane 108 being used to display a second cross-section 112 of the target object 102. In this example, the virtual plane 108 is at an angle relative to the tracking plane 104, thus the auxiliary cross-section 112 is at an angle to the cross section 110. The second cross-section 112 can allow the user 12 to visualize an informative piece of the 3D object 130 that is not seen on the current cross section 110 of the primary plane 104. For example, in medical imaging, the cross-section 112 could be used to provide a second view of an anatomical structure that is also displayed in the cross-section 110 on plane 104. It should be appreciated that the virtual plane 108 may be used to change the display of the target object 102 in any other way, for example displaying the cross section of another 3D object 130 representing the same subject (e.g., CT scan cross-section on tracking plane 104 and MRI cross-section on virtual plane 108). The function of the virtual plane 108 may be selected in the menu 106, or a plurality of virtual planes 108 may be provided in the virtual space 100 with each virtual plane 108 having a different function assigned by the user 12.

In one example, two different functions may be associated with the same virtual plane 108 (not clearly shown). For example, a single plane may simultaneously hide structures while displaying a cross section 112 or model of a 3D object 130 as the virtual plane 108 is moved through the target object 102. Alternatively, one side of the virtual plane 108 (side A), may have a different function from the second side of the virtual plane 108 (side B). In this example, side A may have the function of hiding structures, while side B may have the function of displaying a model of the 3D object 130, such that the orientation of the plane determines what function the plane will serve. In other words, if side A faces to the right and side B faces to the left, as the virtual plane 108 is moved to the right through the target object 102, models on the left (other side of side B) will be illuminated while structures will be hidden on the right (other side of side B). In this example, if the virtual plane 108 was flipped such that side A now faces to the left and side B now faces to the left, structures will be revealed/hidden differently, i.e. as the plane 108 is moved to the right, tumors on the right will be illuminated while structures on the left will be hidden.

In another example, the virtual plane 108 may be assigned to function as the tracking plane 104 by selecting a function to associate the virtual plane 108 dimensions to that of the physical surface 16. In this example, the tracking plane 104 would convert to a virtual plane 108, and the selected virtual plane 108, coupled with the target object 102, would automatically reorient together in the virtual space 100 to align the virtual plane 108 with the physical surface 16, thereby maintaining the cross-section 112 displayed in the process.

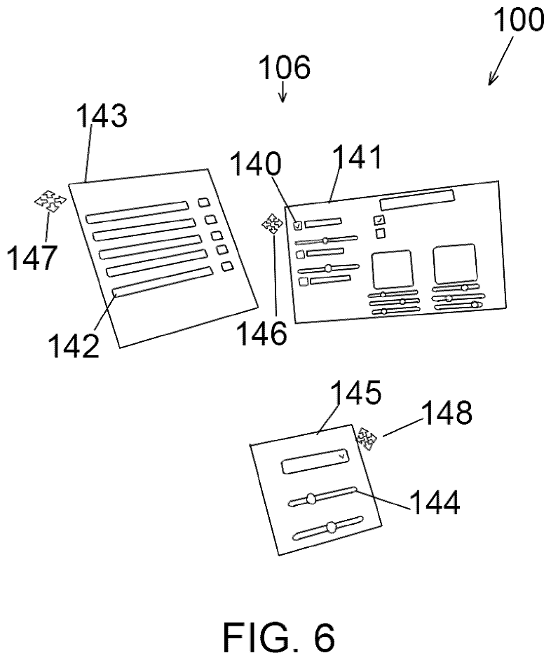

FIG. 6 illustrates the menu 106, comprising one or more panels displayed in the virtual space 100. The user 12 can interact with the menu 106 by moving the virtual stylus 120 or the virtual controller 128 over the menu 106 and toggling or selecting options under the virtual stylus 120 or virtual controller 128 by pressing the stylus button 26 or the controller button 32, respectively. The menu 106 may comprise user interface elements such as one or more checkboxes, one or more buttons, and/or one or more sliders. In this example, the menu 106 comprises a first panel 141 with a plurality of checkboxes 140, a second panel 143 with a plurality of buttons 142, and a third panel 145 with a plurality of sliders 144. The checkboxes 140 toggle the stylus fade and tablet fade effects, as described in greater detail below, the buttons 142 toggle between displaying and hiding the contours 132 around various organs or other structures, and the sliders 144 vary the contrast and brightness of the cross-section 110. It should be appreciated that the menu 106 can take on different forms and display different options to the user 12. The menu 106 can also be displayed, moved, or hidden from view using the virtual controller 128 by pressing one or more controller buttons 32 and/or gestures.

The user 12 can customize the layout of the menu 106 by rearranging or relocating the panels 141, 143, 145. The user 12 can relocate the first panel 141 by moving the virtual controller 128 or virtual stylus 120 over a first relocate option 146, pressing the controller button 32 or the stylus button 26, and physically moving the controller 28 or stylus 20 to move the first panel 141. The user 12 can similarly relocate the second panel 143 by using a second relocate option 147, and the third panel 145 by using a third relocate option 148. As such, the user 12 can customize the placement of the panels 141, 143, 145 such that the checkboxes 140, buttons 142, and sliders 144 are easily accessible.

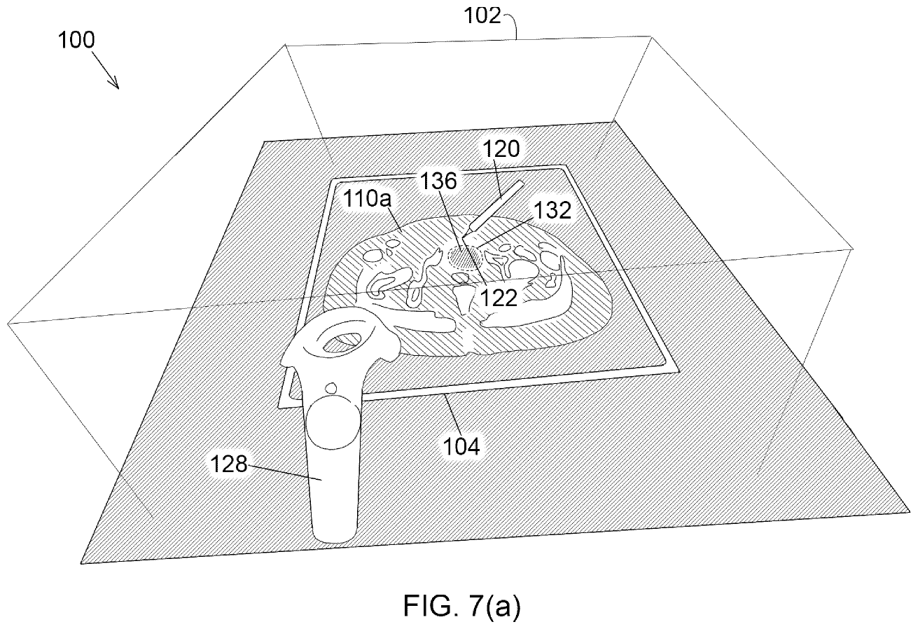

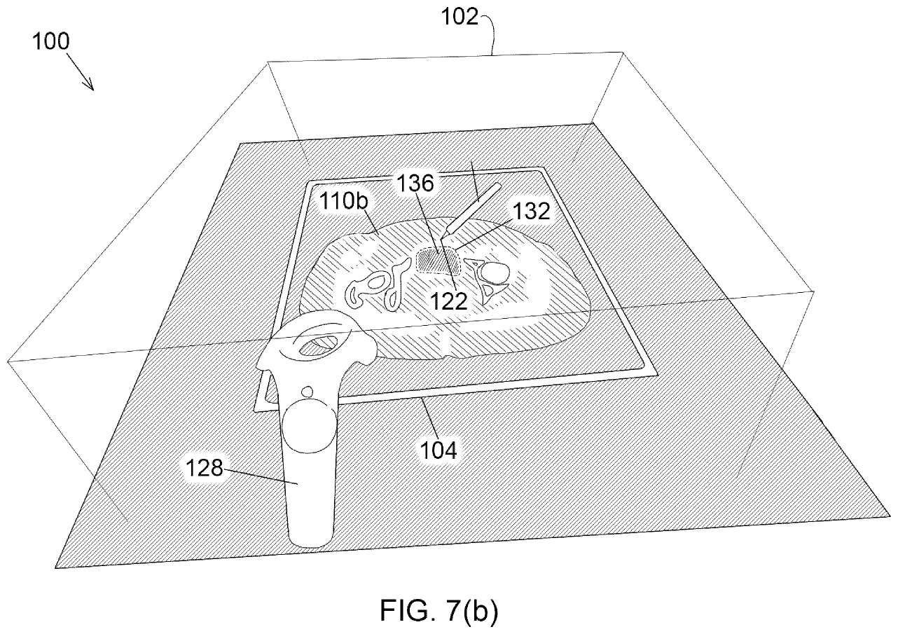

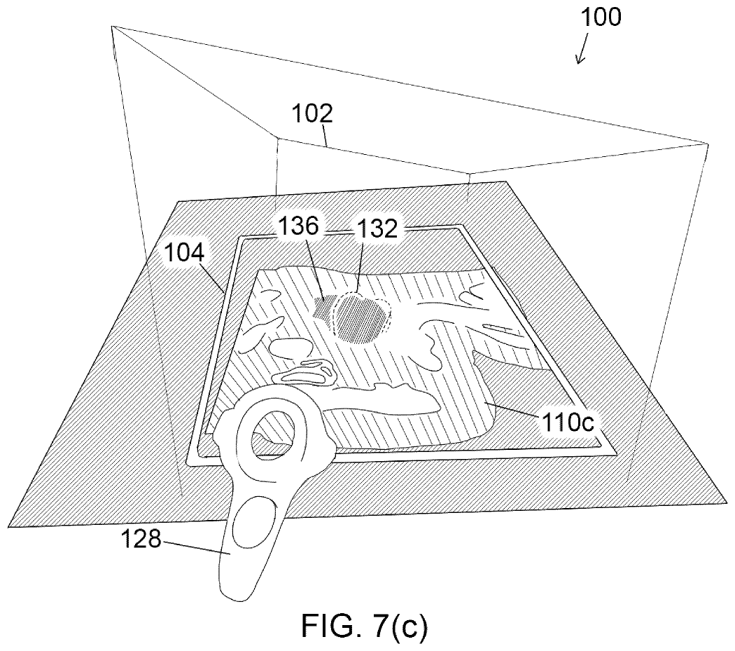

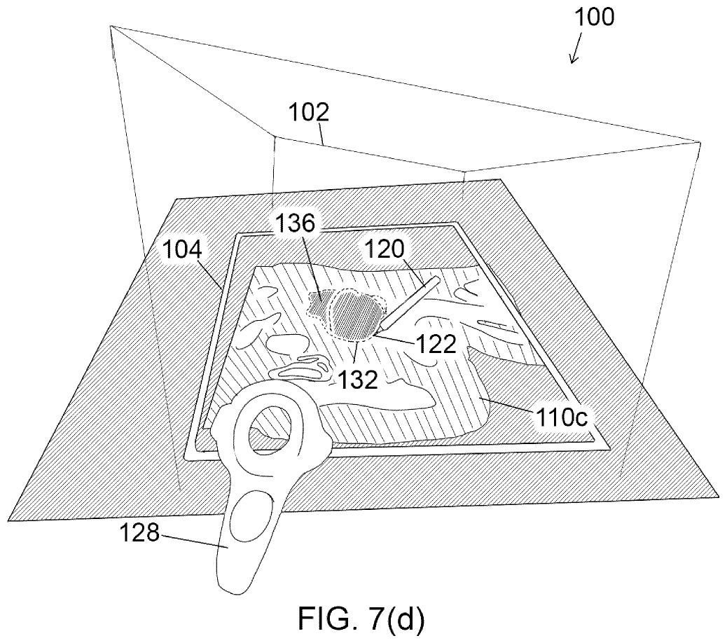

The virtual stylus 120 can be used to apply markings to the target object 102. FIGS. 7(a) through 7(d) illustrate the system 10 being used to mark or otherwise draw an outline around a structure of interest 136. In one example, the structure 136 may be an organ, tumor, bone, or any other internal anatomical structure. Although not shown in FIGS. 7(a) through 7(d), data stored in the target object 102 may be visible when marking the outline around the structure of interest 136. For example, the 3D object 130 and/or the contours 132 may be visible to provide 3D context such as the location of other organs or providing anatomical information that removes ambiguity concerning a region of the cross section 110 currently being examined or contoured. Positional proximity and orientation to other relevant tissue structures can advantageously be visualized in the 3D context.

FIG. 7(a) shows the tracking plane 104 intersecting the target object 102 at a first cross-section 110a. As described above, the virtual stylus 120 can be moved towards the tracking plane 104 by physically moving the stylus 20 towards the surface 16. Upon detecting contact between the stylus 20 and the surface 16, the system 10 is configured to enable markings to be applied using the virtual stylus 120. The system 10 can detect contact between the stylus 20 and the surface 16 using input from the first tracker 22, second tracker 18, and stylus tip sensor 24. For example, if the stylus tip sensor 24 is a button, the system 10 can detect contact when input from the first tracker 22 and second tracker 18 indicates that the stylus 20 is in close proximity to the surface 16, and the button 24 is pressed. In other examples, drawing may be enabled when the stylus button 26 is pressed, allowing the user 12 to begin applying markings when the stylus 20 is not in contact with the surface 16.

When applying the markings by annotating or drawing is enabled by the system 10, the path of the virtual stylus 120 on the tracking plane 104 is marked with a drawn contour 132 until drawing ceases or is otherwise disabled, for example when the user 12 removes the stylus 20 from the surface 16. Once marking is discontinued, the drawn contour 132 may be saved to the target object 102. Marking such as lines, contours and annotations (e.g., measurements, notes, etc.) can also be delineated using a circular paintbrush that fills a region (not shown) rather than a line defining an outline.

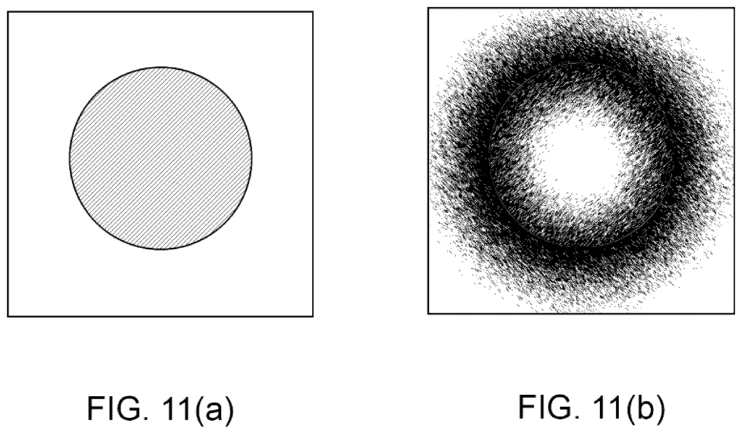

Referring additionally to FIGS. 11(a), 11(b), 12 and 13, further detail concerning applying markings and marking/contouring tools is provided. An objective of radiotherapy contouring (or, similarly, delineation of organs for 3D printing) is to take a volumetric image set and to define a volume within the image set that represents/encompasses a given organ or other anatomical feature. The wireframe planar contours shown in the figures described herein display one particular means of visualization, also referred to as a descriptive mode of these volumes. The wireframe planar contours are a typical storage format for these file types and a typical delineating method in radiation therapy. However, what is of interest for the contouring is not necessarily the wireframe, but the underlying volumes that represent organs or anatomical features.

The following provides further technical details concerning how the presently described VR contouring can be used to change how identified anatomical volumes of interest are defined, while maintaining an intuitive and fluid input mechanism that is familiar to users of traditional systems. Signed Distance Fields (SDFs) are implicit representations of 3D volumes or 2D areas. In a SDF, a 2D grid of pixels or a 3D grid of voxels is defined and for each point, the minimum distance to the surface of the described object is stored as shown in FIGS. 11(a) and 11(b).

SDFs are considered to be useful because they can allow for mathematically simple manipulation of objects (both 2D areas and 3D volumes). For example, constructive solid geometry (CSG) (e.g., when one does “add object A's volume to object B” in CAD software) is done with SDFs. Radiotherapy treatment planning systems use SDFs to interpolate contours that are not defined on every slice: i) currently delineated contours are converted to SDFs, ii) new SDFs are defined on slices without contours by interpolating the SDFs from adjacent slices, then iii) new contours are created on the empty slices by converting the interpolated SDFs into contours. The system 10 can be used for creating contours and object delineations by applying markings via drawing or annotating for example. However, the underlying data structure used can be SDFs.

Using SDFs allows the system 10 to create contours with circular fill paintbrush tools on the primary plane 104. In such an implementation, there is a spherical virtual object attached to the virtual stylus tip 122. When the virtual sphere intersects the tracking plane 104, it defines a circular region on the plane 104. While in a drawing mode, a user sees a “fill-region” contour that is either drawn or erased. In reality, rather than a thick or thin line being drawn, a 2D SDF representing the union (or subtraction for erasing) of all the circular intersection draw positions can be defined. From a user interface perspective, the size of the circular region can be adjusted by holding a stylus button 26 and moving the stylus 20 forwards or backwards. This could also be achieved by a pressure sensing stylus tip sensor 24 or, in another implementation, by pressing the controller button 32 on controller 28 while simultaneously adjusting the size of the circular region by activating the pressure sensing stylus tip sensor 24. It may be noted that the line-drawing mechanism shown herein can still be a useful option for generating a 2D SDF since 2D SDFs can be derived from a closed-loop line contour with a simple and fast algorithm.

The sphere at the end of the virtual stylus tip 122 (or attached to a controller 128) can also be used to define a 3D SDF which now defines a 3D volume (instead of 2D area of the sphere's circular intersection with the plane) in a similar manner. This 3D volume can be visualized with volume rendering or on the tracking plane 104 as a color-fill or contour outline region.

The system 10 can therefore be used to simplify the process for defining a volume that encompasses a given anatomical structure by way of intuitively manipulating 3D SDFs in two ways: (1) using 3D tools to change them directly or (2) using 2D tools to define 2D SDF subsets that describe portions of the final 3D SDF. These 2D SDF subsets (individual contours) are combined to create the resulting 3D SDF volume.

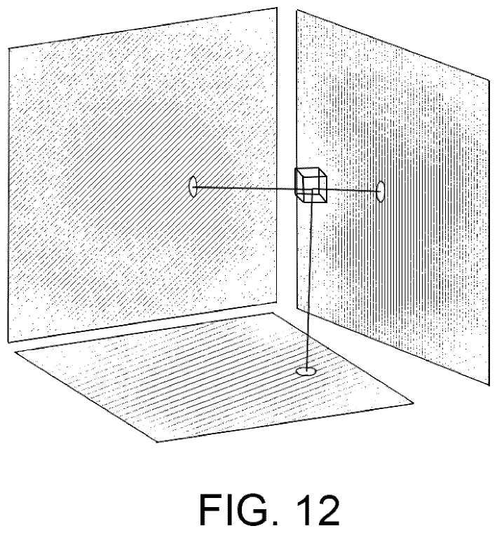

The system 10 can be programmed to take multiple non-parallel 2D SDFs from separate descriptive planes and interpolate them into a 3D SDF that defines the desired 3D structure, as shown in FIG. 12. The workflow discussed above can also be the same in this embodiment. The user 12 rotates and positions the target object 102, contours on that cross section (what appears to be drawing a line or painting a fill region, but in reality is defining a SDF in the background), repositions and contours again, etc. The 3D volume is then created as a result of these 2D SDFs. For example, each voxel in the 3D SDF grid is projected onto the 2D SDFs and the value in that voxel is determined from the values in the 2D SDFs using one of a number of sophisticated interpolation algorithms.

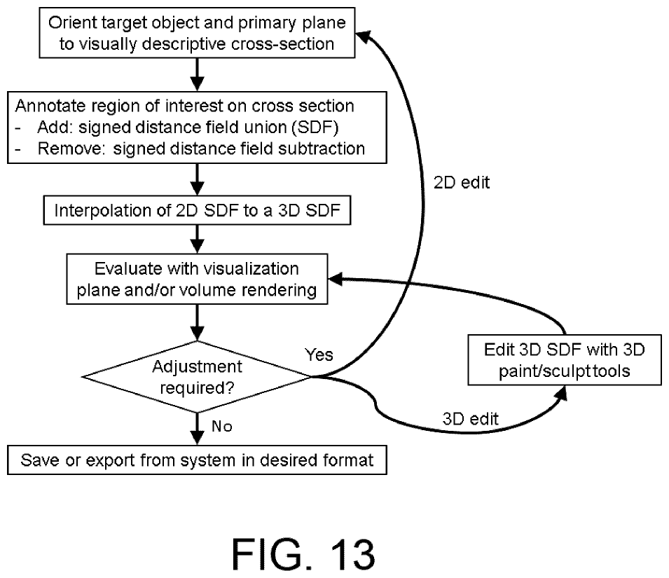

FIG. 13 illustrates an example of a workflow for creating and using 3D SDFs. As shown in FIG. 13, the target object 102 is oriented to a display a visually descriptive, non-parallel cross-section 110 on primary plan 104, and a region of interest (ROI) is contoured on the cross-section 110 as described previously. Constructive solid geometry logic is used to add and subtract marked regions. A 3D reconstruction of 2D SDFs to a 3D SDF is then performed, and this is evaluated with what may be considered 2.5 dimensional (2.5D) visualization and volume rendering. In other words, the 2D planar visualization inhabits a physical 3D space as described above with the use of virtual planes. The user 12 then determines if adjustment is required and, if so, the 3D SDF is edited with 3D paint tools or further 2D SDFs are added to improve the 3D reconstruction and the process is repeated. If no adjustment is needed and the 3D volume is suitably accurate, the volume can be exported from the system 10 in the desired format: for example, a 3D mesh can be generated from the 3D SDF using well known algorithms such as “marching cubes”, or radiotherapy contours can be exported by creating 2D contours on each original image set axial plane position using, for example, the marching squares algorithm.

Returning to FIG. 7(a), if the stylus tip sensor 24 is capable of detecting force, a larger force applied by the stylus 20 on the surface 16 may, for example, result in a larger circular paintbrush radius. As mentioned above, the relative motion of the virtual stylus 120 and the tracking plane 104 corresponds to the relative motion of the stylus 20 and the surface 16. Thus, the path of the stylus tip sensor 24 along the surface 16 is reproduced by the tip of the virtual stylus tip 122 on the tracking plane 104. As such, the user 12 draws a contour 132 of a certain shape on the tracking plane 104 by tracing an identical shape with the stylus 20 on the surface 16. In FIG. 7(a), the contour 132 is created by drawing around the outline of the structure 136 in the first cross-section 110a.

As illustrated by FIG. 7(b), the target object 102 and/or the tracking plane 104 is moved, for example by pressing the controller button 32 configured to incrementally move the target object 102 perpendicular to the tracking plane 104. The target object 102 may be moved such that the tracking plane 104 intersects the target object 102 at a second cross-section 110b. An additional component of contour 132 can be drawn around the outline of the structure 136 in the second cross-section 110b.

The drawn contour 132 can remain visible when the target object 102 is manipulated. In the example shown in FIG. 7(c), the target object 102 is rotated such that tracking plane 104 intersects the target object 102 at a third cross-section 110c, on a different plane and orientation than the first cross-section 110a and the second cross-section 110b. The drawn contours 132 around the outline of the structure 136 in the first cross-section 110a and the second cross-section 110b partially extend from the tracking plane 104. The previously-drawn contours 132 may provide some three-dimensional context for the boundary of the structure 136 when the user 12 draws an additional contour 132 around the outline of the structure 136 in the third cross-section 110c, as shown in FIG. 7(d). Similarly, a partially transparent version of the entire 3D structure derived by interpolating the various 2D contours 132 as described above may remain visible when moving, rotating or re-orienting the contour providing context for the change in orientation.

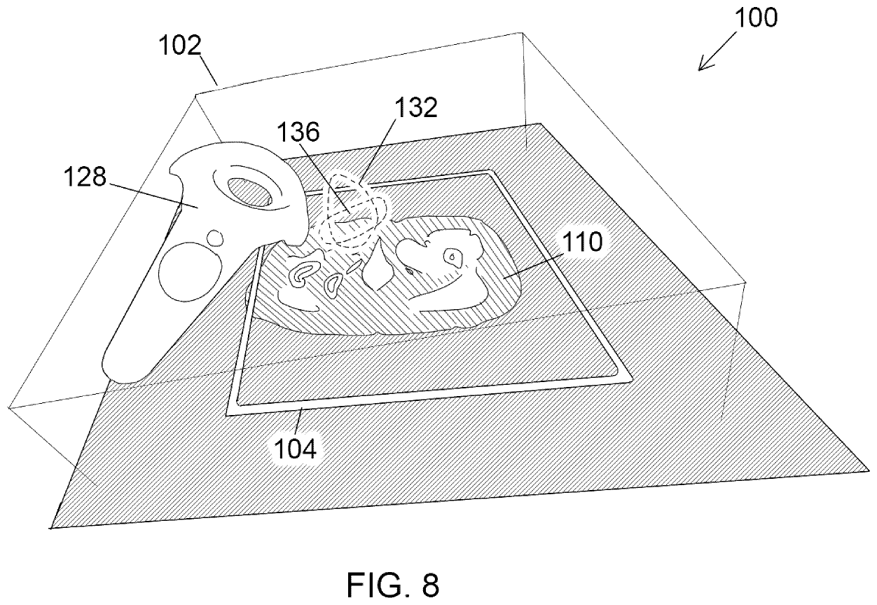

The drawn contours 132 may remain visible when the user 12 continues to manipulate the target object 102, as shown in FIG. 8. As such, the drawn contours 132 can provide an indication of the location and orientation of the structure 136. The color of the drawn contour 132 may be selected in the menu 106, for example to use different colors to distinguish contours 132 around different organs. Similarly, an interpolated 3D volume based on drawn contours may be shown in a distinct color from the contours to distinguish one from the other.

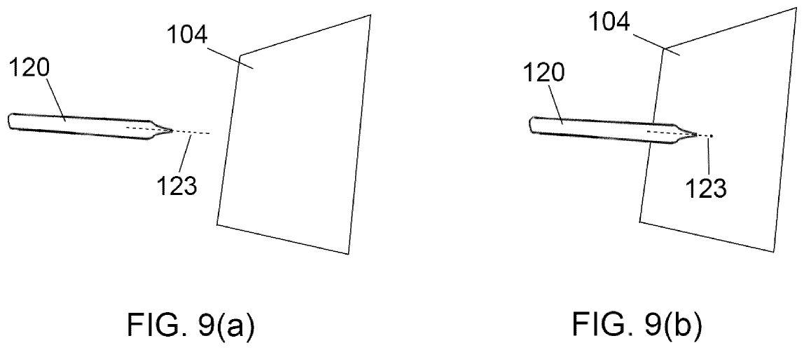

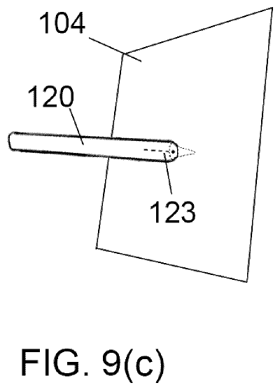

As shown in FIG. 9(a), the virtual stylus 120 may include a virtual tip extension 123. In this example, the virtual tip extension 123 is illustrated as a laser-like beam extending a finite distance beyond the tip of the virtual stylus 120, and a finite distance into the virtual stylus 120. The virtual tip extension 123 can be used to determine and indicate the precise location on the primary plane 104 at which the contour 132 will be applied and alleviate any inaccuracies in the tracking of the stylus 20 and surface 16.

The system 10 may be configured to apply the contour 132 at the point of intersection between the virtual tip extension 123 and the primary plane 104. As illustrated in FIG. 9(b), slight tracking errors may result in the virtual stylus 120 being above the tracking plane 104 even when the stylus tip sensor 24 is in contact with the surface 16. In this example, the tracking plane 104 is within the length of the virtual tip extension 122. As such, the contour 132 is applied on the tracking plane 104, at the point of intersection with the virtual tip extension 123, instead of at the tip of the virtual stylus 120 on a different plane.

Similarly, as illustrated in FIG. 9(c), slight tracking errors may result in the virtual stylus 120 being below the tracking plane 104 even when the stylus tip sensor 24 is in contact with the surface 16. In this example, the portion of the virtual tip extension 123 extending into the virtual stylus 120 intersects the tracking plane 104. As such, the contour 132 is applied on the tracking plane 104, at the point of intersection with the virtual tip extension 123, instead of at the tip of the virtual stylus 120 on a different plane.

The virtual tip extension 123 can additionally be used as a further input to enable an operation that applies markings, e.g. for drawing, annotating, contouring, tracing, outlining, etc. The system 10 may be configured to only enable marking to be applied with the virtual stylus 120 when the tracking plane 104 is within the length of the virtual tip expansion 123, such that the application of markings is not activated when the stylus 20 is away from the surface 16 if the stylus tip sensor 24 falsely detects contact.

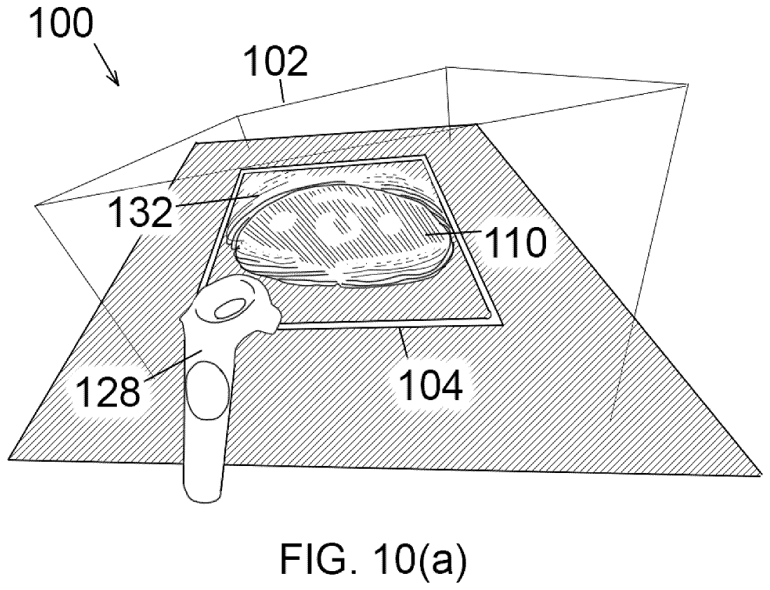

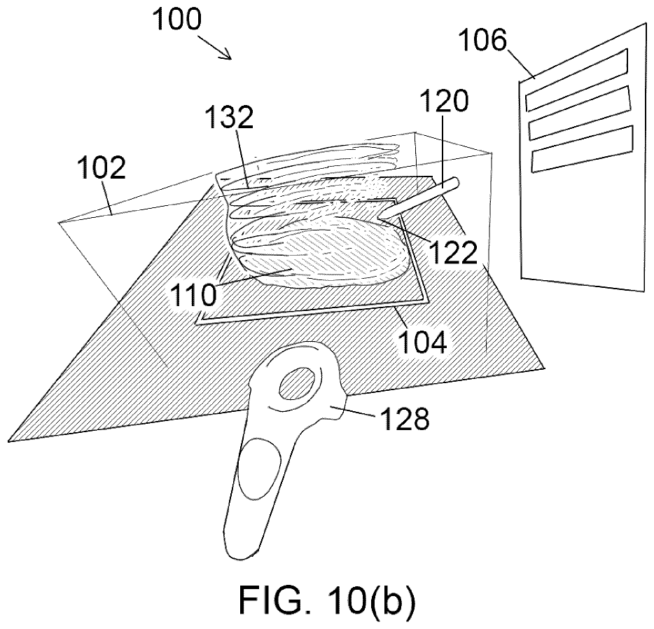

As shown in FIGS. 10(a) and 10(b), the system 10 may be configured to dynamically hide or fade parts of the target object 102, such as the contours 132, creating a transparency gradient. FIG. 10(a) illustrates a surface fade effect, wherein the parts of the target object 102 a distance from the tracking plane 104 are hidden, such that only regions of the target object 102 that are within a certain proximity of the tracking plane 104 are visible. The surface fade effect may be adjusted using options in the menu 106, as described above. The magnitude of the surface fade, or the distance from the tracking plane 104 where the target object 102 begins to fade, may also be selected in the menu 106.

FIG. 10(b) illustrates a stylus fade effect, wherein the parts of the target object 102 near the virtual stylus 120 are hidden. The stylus fade effect may improve the accuracy of the drawn contours 132, as the user 12 does not have their vision of the cross-section 110 occluded by features such as contours 132. The stylus fade effect may be toggled in the menu 106, as described above. The magnitude of the stylus fade, or the amount of the target object 102 around the virtual stylus 120 that is hidden, may also be selected in the menu 106.

In another example, the fade effect may be provided by a virtual flashlight-like object (not shown), which produces a cone of effect, such as the fade effect described above. In one example, the cone of effect may reveal underlying fused scan data (such as PET or MRI) and/or hide other data such as contour 132, wireframes or volumes, or volume rendered image data sets. In one example, the user 12 may reposition the virtual flashlight tool (not shown) and hang it in the virtual space to illuminate the target object 102 from a desired angle.

The system 10 and principles of operation thereof can be adapted to various applications, including both medical and non-medical applications.

For example, the system 10 can be used, in any such application for human subjects, virtual characters, or animals. That is, the system 10 can be used in a virtual space 100 that is other than that intended for radiation oncology contouring. For example, surgical planning, gaming, drawing/editing, design, communication/conversing, etc.

In one example, the system 10 can be adapted to provide a drawing application for learning how to draw 3D figures and/or as a means/method for drawing in 3D, using the assistance of the tracking plane 104 corresponding with the volume rendered. This would be useful for people designing games or any 3D object such as furniture design or 3D printing files. It may be noted that, in a “learn to draw” scenario, the 3D volume rendered would be the object being studied, and the user 12, for example a student, can trace the image along different chosen axes or planes to arrive at the final 3D structure.

In another example, the system 10 can be adapted to provide an application for engineers or architects working on 3D building plans or structures, or similarly for 3D printing. For example, a user 12, for example an architect or engineer, could have a 3D blueprint of their plans as the virtual image in the virtual space 100, and any changes they want to make at any place can be accomplished by moving the tracking plane 104 to the desired position to write and at which angle to sketch amendments. In this application, the user 12 could also snap the view to automatically rotate and reorient the image file so that the chosen plane is now mirroring the physical surface 16 and they can draw on it easily in order to manipulate the plans. This can similarly be done for 3D printing structure designs.

In yet another example, a tool for interior design and/or landscape design can be provided, wherein the user 12 can annotate walls/furniture or shrubs/trees by writing notes wherever desired, by inserting a tracking plane 104 into the virtual space 100 that corresponds with a writing surface, or cutting (instead of drawing, make it a selection tool to cut parts out) and moving elements around the virtual space 100.

Further to the above examples, another application includes adapting the system 10 described above for teaching purposes or other collaborative purposes, both networked (live) and asynchronous (recorded), additionally utilizing audio features including microphones for recording and speakers for audio playback. For example, the user 12 drawing can be conducting a lecture/illustration to show one or more students/attendees parts of an anatomy or how to target or not target for radiation therapy. This can be done asynchronously (pre-recorded) or networked (live). All users 12 (e.g. teachers and viewer students) can be placed in the virtual space 100 regardless of whether the lecture/lesson is pre-recorded or live. If the lesson is asynchronous, the viewer can pause the lesson, and move closer to, manipulate, move or otherwise inspect 3D objects around in the virtual space 100. When un-paused, the lesson may revert to a previous setting and the viewing user continues to watch the lecture. In one embodiment, the system 10 can be configured such that if the viewing user 12 draws on or annotates a 3D object 130 when the system is “paused”, the drawing can remain while the user 12 continues to watch the lecture so that the user can see how it fits with the rest of the lecture. In another embodiment, the user 12 can make notes on the lecture that appear and disappear throughout and can save these annotations. In yet another embodiment, teaching can be done so that the student user 12 can practice drawing, and if that user veers off or is wrong, the system 10 can notify the user 12. This can be done in real time, with the system 10 generating alerts as the user 12 makes errors, and can be done as a tutorial where the system 10 guides the user 12 and provides tips as the user 12 progresses through an exercise, or can be done as a test, where the user 12 attempts a contour and when completed, receive a score or other evaluation of performance.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

It will also be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the testing tool 12, any component of or related to the computing environment 10, etc., or accessible or connectable thereto. Any application or module herein described may be implemented using computer readable/executable instructions that may be stored or otherwise held by such computer readable media.

Although the above principles have been described with reference to certain specific examples, various modifications thereof will be apparent to those skilled in the art as outlined in the appended claims.

1. A system for applying markings to a three-dimensional virtual image or virtual object using a surface, the system comprising:
an object capable of being used as a physical stylus; and
a virtual or augmented reality display;
wherein a virtual space is displayed by the virtual or augmented reality display, the virtual space comprising:
a three-dimensional target object;
at least one plane, including a tracking plane, the tracking plane corresponding to the surface; and
a virtual stylus in a virtual reality view, or the physical stylus in an augmented reality view; wherein:
a position of the virtual stylus or the physical stylus relative to the tracking plane is correlated to an actual position of the physical stylus relative to the surface; and
a cross-section of the target object is displayed on the tracking plane where the tracking plane intersects the target object.

2. The system of claim 1, wherein the system is configured to determine the actual position of the physical stylus relative to the surface using a tracking system associated with the surface and the physical stylus.

3. The system of claim 2, wherein the tracking system comprises one or more trackers provided on the surface and the physical stylus.

4. The system of claim 2, wherein the tracking system comprises one or more calibrated positions in the virtual space.

5. The system of claim 1, wherein the physical stylus is provided with a stylus tip sensor.

6. (canceled)

7. The system of claim 1, wherein the system further comprises a controller.

8. (canceled)

9. The system of claim 7, wherein the virtual space further comprises a virtual controller, and a movement of the virtual controller is associated with a movement of the controller.

10. The system of claim 9, wherein the target object is moved by moving the virtual controller onto the target object, pressing a first controller button of the controller, and moving the virtual controller while holding the first controller button.

11. (canceled)

12. (canceled)

13. The system of claim 1, wherein the system is configured to:
detect contact between the physical stylus and the surface;
delineate a region of interest using a circular or spherical fill region; and
save the region of interest to the target object upon detecting a break on the contact between the physical stylus and the surface.

14. The system of claim 1, wherein the system is configured to:
detect contact between the physical stylus and the surface;
mark a region of interest along a path of the virtual stylus while detecting contact between the physical stylus and the surface; and
save the region of interest to the target object upon detecting a break in the contact between the physical stylus and the surface.

15.-18. (canceled)

19. A method of applying markings to a three-dimensional virtual image or virtual object, the method comprising:
displaying a virtual space using a virtual or augmented reality display;
providing in the virtual space: a three-dimensional target object, at least one plane including a tracking plane, the tracking plane corresponding to the surface, and a virtual stylus in a virtual reality view, or an object used as a physical stylus in an augmented reality view;
correlating a position of the virtual stylus or the physical stylus relative to the tracking plane to an actual position of the physical stylus relative to a surface; and
displaying a cross-section of the target object on the tracking plane where the tracking plane intersects the target object.

20. The method of claim 19, further comprising determining the actual position of the physical stylus relative to the surface using a tracking system associated with the surface and the physical stylus.

21. The method of claim 20, wherein the tracking system comprises one or more trackers provided on the surface and the physical stylus.

22. The method of claim 20, wherein the tracking system comprises one or more calibrated positions in the virtual space.

23. The method of claim 19, wherein the physical stylus is provided with a stylus tip sensor.

24. (canceled)

25. The method of claim 19, wherein the method utilizes a controller.

26. (canceled)

27. The method of claim 25, wherein the virtual space further comprises a virtual controller, and a movement of the virtual controller is associated with a movement of the controller.

28. The method of claim 27, further comprising moving the target object by moving the virtual controller onto the target object, detecting pressing a first controller button of the controller, and moving the virtual controller while the first controller button is being held.

29. (canceled)

30. (canceled)

31. The method of claim 19, further comprising:
detecting contact between the physical stylus and the surface;
delineating a region of interest using a circular or spherical fill region; and
saving the region of interest to the target object upon detecting a break on the contact between the physical stylus and the surface.

32. The method of any one of claims 19 to 30, further comprising:
detecting contact between the physical stylus and the surface;
marking a region of interest along a path of the virtual stylus while detecting contact between the physical stylus and the surface; and
saving the region of interest to the target object upon detecting a break in the contact between the physical stylus and the surface.

33.-36. (canceled)

37. A non-transitory computer readable medium comprising computer executable instructions for:
displaying a virtual space using a virtual or augmented reality display;
providing in the virtual space: a three-dimensional target object, at least one plane including a tracking plane, the tracking plane corresponding to the surface, and a virtual stylus in a virtual reality view, or an object used as a physical stylus in an augmented reality view;
correlating a position of the virtual stylus or the physical stylus relative to the tracking plane to an actual position of the physical stylus relative to a surface; and
displaying a cross-section of the target object on the tracking plane where the tracking plane intersects the target object.
